(19) United States
(12) Patent Application Publication
(10) Pub. No.: US 2024/0238608 A1
(43) Pub. Date: Jul. 18, 2024

(54) Neural Electromagnetic Transduction, Modulation, and Catalysis

(71) Applicant: Hector Humberto Gomez Acevedo, Mexico City (MX)

(72) Inventor: Hector Humberto Gomez Acevedo, Mexico City (MX)

(21) Appl. No.: 18/538,323

(22) Filed: Dec. 13, 2023

Related U.S. Application Data
(60) Provisional application No. 63/495,296, filed on Apr. 10, 2023.
(60) Provisional application No. 63/387,695, filed on Dec. 15, 2022.

(51) Int. Cl. A61N 2/00; A61N 2/02

(52) U.S. Cl. CPC A61N 2/006; A61N 2/02

(57) ABSTRACT

The present disclosure teaches a magnetic field technology that generates longer and more concentrated magnetic density, thereby enabling the selection and stimulation of specifically planed neural circuits to produce plasticity and regeneration of biological material (e.g., cellular structures) by inhibiting the neural circuits that inhibit the plasticity and regeneration of such benefic neural circuits.

### RELATED APPLICATIONS

This application claims the benefit of U.S. Provisional Application Ser. No. 63/387,695, filed Dec. 15, 2022, titled NEURAL ELECTROMAGNETIC TRANSDUCTION AND MODULATION, and U.S. Provisional Application Ser. No. 63/495,296, filed Apr. 10, 2023, titled NEURAL ELECTROMAGNETIC TRANSDUCTION, MODULATION, AND CATALYSIS, the entire disclosures of which are herein incorporated by reference.

### BACKGROUND

### Technical Field

The present disclosure generally relates to neuromodulation. More particularly, but not exclusively, the present disclosure relates to systems, devices, and methods for neural electromagnetic transduction and modulation.

### Description of the Related Art

It is known that localized magnetic fields (LMFs) could easily penetrate a patient's hair, scalp, skull, meninges, cerebral spinal fluid (CSF), and brain thereby inducing an electrical current in the central and peripheral nervous systems. It is known that LMFs are primarily used in transcranial magnetic stimulation (TMS) to cause or at least encourage certain effects on different regions or cells of the brain and its activities. Certain therapeutic TMS approaches are known.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which, in and of itself, may also be inventive.

### BRIEF SUMMARY

The following is a summary of the present disclosure to provide an introductory understanding of some features and context. This summary is not intended to identify key or critical elements of the present disclosure or to delineate the scope of the disclosure. This summary presents certain concepts of the present disclosure in a simplified form as a prelude to the more detailed description that is later presented.

Electromagnetic modulation of a patient's nervous system may be understood as the functional change obtained on such system in response to electric or magnetic stimulation. For decades transcranial magnetic stimulation (TMS) has been used for certain medical therapies including to ameliorate depression. The results of TMS therapies are in some cases good but limited. One reason for the limited results is believed to be related to the application of such energy only on the reachable, superficial regions of the brain's frontal lobes that most electromagnets can stimulate. Notably, even when deeper regions of the patient's brain are forced to interact with stronger electromagnetic fields or different electromagnet shapes, the stimulation is very sparse and unable to be applied selectively amongst neighboring small regions, thus making very difficult to purposely stimulate or inhibit a specific circuit alone.

The present disclosure teaches a magnetic field technology, including but not limited to an advanced array of electromagnets based on iterative solenoids arrays, that generates longer and more concentrated (i.e., denser) harmonic magnetic fields, thereby enabling the selection and stimulation of specifically planed neural circuits to produce plasticity and regeneration of biological material (e.g., cellular structures) by inhibiting the neural circuits that inhibit the plasticity and regeneration of such benefic neural circuits.

This technology may be used to transfer magnetic power over anatomical regions as well as molecular vibration and heat over selectively chosen molecular types within such regions in order to enhance biochemical reactions (i.e., catalysis). For example, this technology may be used to inhibit the craving for opioids, food, and alcohol by magnetically disrupting magnetic signals and normal electro-biochemical signaling of the craving feeling mediated by a hypertrophied neural circuit comprised by the supra-orbital cortex of the brain as well as the accumbens nucleus. Such amelioration of the craving sensation has been discovered by the inventor to release from their inhibition the self-control neural circuit comprised by the prefrontal cortex and the sub-thalamic nucleus. Toward this end, the multiple signaling variants provided by the iterative solenoids arrays and their harmonic electromagnetic fields provide an improved input and output data transducer, and variants on the embodiments of this device also employ the catalysis technology for the therapeutic modulation of the biochemical reactions on human tissues such as the neuronal parenchyma.

In more detail, the technology presented in the present disclosure can be used for many purposes, including to inhibit the supra-orbital cortex of the patient's brain as well as the Accumbens Nucleus (i.e., nucleus accumbens, NAc, NAcc, nucleus accumbens septi, or the like). Such inhibiting properties are targeted for various reasons including the reason that those structures are known to produce, induce, or otherwise act in the creation of cravings for opioids and food. Hence, by subjecting the NAc to certain TMS as taught herein, such structures may be weakened, and one result of such weakening is believed or otherwise expected to include the reduction or other amelioration of the craving sensation. In more detail, by subjecting the NAc to certain TMS as taught herein, those particular circuits will stop to overwhelm and to weaken the prefrontal cortex as well as the subthalamic Nucleous given that these structures work to produce a “Don't Go” signal against the craving. On the other hand, electric stimulation is unable to fully interact with the information that is transmitted through a tract with just two to four (2 to 4) electrodes that generate an electric field. The present inventor has come with additional technology to better transduce such information.

In an embodiment, a novel neuromodulation device can employ Far-Field Magnetic Transfer effect and it can include at least a pair of iterative multiple-magnet arrays wherein each array can include a number of magnets consecutively nested into a bigger one with seemingly parallel walls in a multilayered fashion in order to better produce said effect. The novel neuromodulation device can employ Far-Field Magnetic Transfer technology including at least a pair of iterative multiple-solenoid arrays wherein each array can include a number of solenoids that are independently electrically fed and consecutively nested into a bigger one with seemingly parallel loops. An emitter array and the receiver arrays of the neuromodulation device are coupled to better produce the Far-Field Magnetic Power Transfer effect because each emitter solenoid is electrically fed with the same electrical signal frequency than its equivalent layer solenoid on the receiver multilayered array. Both arrays of the neuromodulation device can contain a metallic core. Such metallic core may be composed of iron, Permalloy, Mu-Metal or any metallic alloy with enough magnetic permeability to increase inductance. The magnetic power of the neuromodulation device can be applied directly to neuronal tissue in order to alter the electric signaling of neural circuits such as the Supra-Orbital cortex with the Accumbens Nucleus circuit. The application of the magnetic power can be applied directly to neuronal tissue in order to stimulate the electric signaling of a neural circuit as the Prefrontal cortex with the Subthalamic Nucleus circuits. The emitter array and the receiver arrays can be electrically connected in serial fashion and the solenoids on each array can be connected in electrically parallel fashion in order to very easily and un-expensively control the device parameters. Each emitter solenoid can be electrically fed independently in order to send multiple signal variations to the solenoids, consequently producing a complex multi emitter magnetic field in order to generate a complex data input transducer capable of interacting with a much bigger neuronal population in a concentrated region of the brain. Each receiver solenoid can be electrically independently connected in order retrieve the signal variations collected by each solenoid of the receiver array, in order to generate a complex data output transducer capable of sensing a much bigger neuronal population in a concentrated region of the brain. A catheter inserted on the brain includes at least two mini multi-solenoids arrays in order to produce a Multi Emitter Magnetic Field in a particular region of the brain. A catheter inserted on the brain comprises at least two mini multi-solenoids arrays deploys an helicoidally shaped coil that emerges from its deeper end and it is introduced through parenchyma by rotatory motion where such catether contains at least two multi solenoid arrays in order to produce a Multi Emitter Magnetic Field in a wider and helicoidally shaped way. The multi solenoid emitters magnetic field produced by the device, in combination with the application of electromagnetic radiative energy, can produce selective molecular type heating and molecular vibration and twisting in order to enhance a desired selective atomics as well as molecular chemical reaction. The multi solenoid emitters magnetic field produced by the device, in combination with the application of electromagnetic radiative energy, can produce selective molecular type heating and molecular vibration and twisting in order to enhance a desired molecular therapeutic biochemical reaction. The multi solenoid emitters magnetic field produced by the device, in combination with the application of electromagnetic radiative energy, at the Larmor's frequency of the target atom can produce selective atomic heating and vibration in order to enhance a desired molecular therapeutic biochemical reaction. The multi solenoid emitters magnetic field produced by the device, in combination with the application of electromagnetic radiative energy, at the Larmor's frequency of the target molecule can produce selective molecular heating, vibration and twisting in order to enhance a desired molecular therapeutic biochemical reaction.

This Brief Summary has been provided to describe certain concepts in a simplified form that are further described in more detail in the Detailed Description. The Brief Summary does not limit the scope of the claimed subject matter, but rather the words of the claims themselves determine the scope of the claimed subject matter.

### BRIEF DESCRIPTION OF THE SEVERAL VIEWS OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIG. 1 is an embodiment of a novel neuromodulation system and method formed with particular electromagnet arrays;

FIG. 2 is an embodiment of a multi-electromagnetic device useful for functional transcranial magnetic stimulation;

FIG. 3 is an embodiment of an intracranial electromagnetic array for intra parenchymal, endovascular and endocavity neuromodulation, and magnetic, electric, and/or electromagnetic transduction;

FIG. 4 is an embodiment of a transcranial magnetic stimulation device that employs far field technology;

FIG. 5 is another intracranial electromagnetic array 100e embodiment for intra parenchyma, endovascular, and endocavity neuromodulation magnetic transduction; and

FIG. 6 is an array embodiment of neuromodulation iteratively shaped solenoids where a multisource magnetic field is employed concurrently with electromagnetic radiation on the frequency range of radio and microwaves to produce atomic and molecular catalysis.

### DETAILED DESCRIPTION

The present disclosure may be understood more readily by reference to this detailed description and the accompanying figures. The terminology used herein is for the purpose of describing specific embodiments only and is not limiting to the claims unless a court or accepted body of competent jurisdiction determines that such terminology is limiting. Unless specifically defined in the present disclosure, the terminology used herein is to be given its traditional meaning as known in the relevant art.

In the following description, certain specific details are set forth in order to provide a thorough understanding of various disclosed embodiments. However, one skilled in the relevant art will recognize that embodiments may be practiced without one or more of these specific details, or with other methods, components, materials, and the like. In other instances, certain structures associated with neural electromagnetic transduction and modulation such as computing devices, control systems, wired and wireless communications protocols, wired and wireless transceivers, radios, communications ports, user interface devices and certain algorithms have not been shown or described in detail to avoid unnecessarily obscuring more detailed descriptions of the embodiments.

Electric neuromodulation may be achieved by the simple electric discharge of electrodes placed on the scalp, sometimes on the temporal areas as an anti-depressive tool. A refinement of the electric stimulation is the use of a multiple poles electrode that is placed in contact with the dura mater of the medullae or nerve roots, within the spinal canal for pain control. Another advancement of electric neuromodulation, which may also be referred to as deep brain stimulation (DBS), includes the placement of a small probe with multiple electrodes, usually within the boundaries of the sub-thalamic nucleus or the geniculate nucleus, with the purpose to ameliorate spasticity and tremor on neurologic conditions such as Parkinson's disease, multiple sclerosis, or the like. One problem with this conventional technology, however, is that the electric field formed by the electrodes is unable to interact in an orderly way with the affected neural structure. Another problem is that this conventional technology produces electric discharges through random micro routes defined by local impedances. Such restrictions prevent the proper electric signal transduction on both, input/output modalities. Therefore, interaction is frequently limited to a combination of electrodes that best suits some desired response, combined with variations of voltage, frequency, and impedance as well as electrical signal amplitude and shape.

Recently, some variations on the shape of the electric field obtained from the electrode's orientation have been reported as an advance. So, as can be observed by anyone familiar with the complexity of neural physiology, the expected results have been limited to a few small achievements mostly related to the discovery of possible new stimulation targets within the brain's anatomy. The present inventor has recognized that technologically meaningful transduction upgrades have been lacking for at least the last 25 years. The same limitations as in earlier conventional technologies were found even with multiple electrodes as in the case of the device known as NEUROLINK, given that the placement of about a dozen electrodes through the cortex of the brain is unfortunately still insufficient to mimic the data acquisition protocol displayed by hundreds of thousands of neurons on the area of interaction.

Another device that is manufactured by SYNCHRONOUS works as a simple electrical electrode for the electric signals recollection produced on a motor cortex. The SYNCHRONOUS device is formed in the shape of a net of about eight millimeter (8 mm) long and that is deployed using an endovascular route within a vessel on the neighborhood of the motor cortex as if it were an electrically wired stent, but the SYNCHRONOUS device suffers the same limitations as were found with NEUROLINK and other devices. In operation, according to which part of the body the patient intents to move, the SYNCHRONOUS device receives a different signal through the wire into an implanted processing unit and transmits the signal to an external computer. There is no neuromodulation involved in the SYNCHRONOUS system, but this system is mentioned because the novel technology taught in the present disclosure should not be confused with such device.

Magnetic modulation of a patient's brain can be achieved with the use of TMS stimulators. There are three basic types of TMS: single-pulse, paired pulse, or repetitive TMS (rTMS) which is in some cases associated with BRAINSWAY. In some cases, a TMS device does deep Transcranial Magnetic Stimulation (Deep TMS). Deep TMS conventionally uses a specially designed “H-shaped” coil that is housed inside a helmet. This design is intended to deliver stimulation deeper in the brain.

Certain transcranial magnetic stimulation (TMS) systems have been cleared by the United States Food and Drug Administration (FDA) to treat major depression and for additional indications. TMS therapy systems (also called rTMS) are sometimes distinguished by: 1) usability features that streamline the therapy experience for practitioners and patients; 2) the availability of different therapy waveforms which are associated with treatment times; and 3) the availability of a neuro-navigated option for patients with magnetic resonance images (MRIs). A TMS session could last anywhere from three to thirty-seven minutes (3 min. to 37 min.) An rTMS session takes about nineteen minutes (19 min.) with newer TMS devices and about thirty-seven minutes (37 min.) with older TMS devices. Conventional deep TMS sessions take about twenty minutes (20 min.).

Intermittent theta burst stimulation (iTBS) has been used as a neural modulation approach to stroke rehabilitation. iTBS is a specific form of repetitive TMS (rTMS) that enhances cortical excitability and modulates oscillatory dynamics in the stimulated area and remote brain regions. An iTBS session takes about three minutes (3 min.) for regular theta-burst stimulation and about nine minutes (9 min.) for an accelerated TMS protocol.

There are different types of known TMS machines. Since the release of the NEUROSTAR, additional TMS devices have gained FDA approval for depression treatment including: APOLLO, BRAINSWAY DEEP, CLOUDTMS, MAGSTIM, MAGVENTURE, NEUROSTAR, and NEXSTIM. Unfortunately, little research has been done to compare these devices and their clinical effectiveness.

Magnetic stimulation can be used as Transcranial magnetic stimulation (TMS). Here, a noninvasive procedure uses magnetic fields to stimulate nerve cells in the brain to improve symptoms of depression. TMS is typically used when other depression treatments haven't been effective.

Some devices are used for TMS. For instance, BRAINSWAY TMS device does deep TMS. Deep TMS uses a specially designed “H-shaped” coil which is housed inside a helmet. This design is intended to deliver stimulation deeper in the brain.

Two types of magnetic therapy are static magnet therapy and electromagnetic therapy. Static magnets vary in strength from about 300 gauss to about 5000 gauss.

Electromagnetic therapy has been used to promote bone healing, wound healing, sleep, and other medical needs. NEURONETICS is a company that markets NEUROSTAR ADVANCED THERAPY. In 2008, NEUROSTAR was the first TMS treatment to be cleared by the FDA for the treatment of depression. Since that time, millions of NEUROSTAR ADVANCED THERAPY TMS treatments have been performed in thousands of patients. Antidepressant medications and psychotherapy have helped alleviate the symptoms of depression for millions of people, but these methods are not successful for all patients. Some patients experience intolerable side effects from the medications, and for some, the medications and psychotherapy treatments do not work at all.

Transcranial magnetic stimulation (TMS) is a non-invasive, drug free alternative treatment for major depressive disorder. Studies have shown that as many as sixty percent (60%) of patients with treatment-resistant depression experience an improvement of their symptoms with TMS therapy, and for about one-third of those patients, TMS eliminates their symptoms completely. The results are not permanent, but even a few months of relief can make a significant difference in a patient's quality of life.

Electromagnets are deployed in a wide variety of use cases. Electromagnets are used in generators, motors, transformers, electric buzzers, bells, headphones, loudspeakers, relays, valves, data storage devices (e.g., video cassette recorders (VCRs), tape recorders, hard discs, induction cookers, magnetic locks, MRI machines, particle accelerators, mass spectrometers, and many others. Many electric appliances used in the home use electromagnetism as a basic working principle. Some electromagnet uses in the home include electric fans, electric doorbells, induction cookers, magnetic locks, and many more. In an electric fan, for example, electromagnetic induction keeps the motor and fan blades rotating. Also, in an electric doorbell when the button is pressed, due to the electromagnetic forces, a coil gets energized and the bell sounds.

The uses of electromagnets are also seen in the medical field. A magnetic resonance imaging machine (MRI) is a device that uses electromagnets to generate images of a patient's internal biological material. The MRI device can scan all the tiny details in the human body with the help of electromagnetism.

Electromagnetism is used in memory storage devices and computer hardware. The data in e-books, gadgets, phones, and many other computing devices are stored in the electromagnetic format and represented as bytes and bits. The computer hardware may also have a magnetic tape, which works on the principle of electromagnetism. Even in the golden days, electromagnets had a huge role in the data storage of VCP and VCR. Without electromagnets, the devices (e.g., telephones, mobile phones, and the like) used to make phone calls over a long distance could not have taken shape. The electromagnetic pulses and the interaction of the signals are what enable modern communication devices to operate.

NEUROSTAR TMS is marketed by NEURONETICS. NEURONETICS sponsored a pivotal trial published in December 2007. In October 2008, the FDA approved TMS Therapy using the NEURONETICS NEUROSTART ADVANCED THERAPY brain-stimulating device for patients suffering from major depressive disorder and who have failed to receive satisfactory improvement from antidepressant medication in the current episode. Being the first device to receive FDA approval in the US for depression treatment gave NEURONETICS “first mover” advantage in penetrating the US market. NEURONETICS went on to pioneer clinical TMS practice in the US including a pivotal role in advocating for insurance coverage across the country. NEURONETICS was also the first to receive FDA clearance for a nineteen minute (19 min.) treatment time.

NEURONETICS NEUROSTAR ADVANCED THERAPY uses a figure-8 coil. The system at the time of this disclosure has three known treatment variations: 1) standard, thirty-seven-and-one-half minutes (37.5 mins.) per session; DASH, nineteen minutes (19 mins.) per session, and TouchStar, which is an iTBS protocol. The NEURONETICS NEUROSTAR ADVANCED THERAPY system does not use a navigation system for MRI guided placement of the coil. Instead, the NEURONETICS NEUROSTAR ADVANCED THERAPY system has a special contact sensing strip that will alert the medical practitioner if contact between the coil and the patient's head is not good.

NEURONETICS has a “pay-per-click” business model, which means that the TMS clinic has to effectively pay a fee for every session.

BRAINSWAY is based in Israel but does many clinical trials in the USA. The BRAINSWAY TMS device does deep TMS. Deep TMS uses a specially designed “H-shaped” coil which is housed inside a helmet. This design is intended to deliver stimulation deeper in the brain. Deep TMS coils do not work with any neuro-navigation systems.

BRAINSWAY FDA clearances and treatment are typically linked to specific coil designs. The BRAINSWAY HI coil is FDA-cleared for treatment resistant depression. The BRAINSWAY H7-coil is FDA-cleared for obsessive compulsive disorder (OCD). The BRAINSWAY DEEP TRANSCRANIAL MAGNETIC STIMULATION SYSTEM H4 is FDA-cleared for helping short-term smoking cessation.

MAGVENTURE is based in Denmark. MAGVENTURE provides a range of systems to support the research market as well as clinical systems. MAGVENTURE TMS Therapy was used among the largest clinical trials with TMS to date (Blumberger et al, 2018, The Lancet: “THREE-D: a randomized non-inferiority trial”), comparing the standard, thirty-seven minute (37 min.) TMS protocol to a newer three minute (3 min.) Theta Burst protocol. The study concluded that the Theta Burst protocol is as safe and effective for the treatment of major depressive disorder as standard TMS.

In 2018, MAGVENTURE received FDA clearance for the three minute (3 min.) protocol marketed as EXPRESS TMS. MAGVENTURE is also FDA cleared for the standard thirty-seven minute (37 min.) protocol as well as the nineteen minute (19-min.) protocol. The MAGVENTURE COOL D-B80 coil is FDA-cleared for adjunctive treatment of OCD.

NEUROSOFT CLOUDTMS, made by Russia-based NEUROSOFT, markets rTMS systems as CLOUDTMS in the US. The CLOUDTMS system has received FDA clearance for thirty-seven-and-one-half minutes (37.5 mins.) per session and nineteen minutes (19 mins.) per session treatment for major depressive disorder. The CLOUDTMS system is not neuro-navigated, but it can be used with a SOTERIX MEDICAL system.

MAG & MORE APOLLO TMS is a therapy system developed and marketed Germany-based MAG & MORE. The MAG & MORE APOLLO TMS uses a marketed HANS positioning system and a touch based patient management system. The MAG & MORE APOLLO TMS system has received FDA clearance for the thirty-seven-and-one-half minutes (37.5 mins.) per session and nineteen minutes (19 mins.) per session treatment for major depressive disorder.

NEXSTIM is based in Finland. The SMARTFOCUS system by NEXSTIM was originally developed to support neurosurgeons with brain mapping before performing brain surgery. A version of the NEXSTIM SMARTFOCUS system is FDA cleared for depression therapy. NEXSTIM TMS is capable of rTMS, iTBS, and neuronavigation TMS which is marketed as NBT Navigated Brain Therapy.

Medical engineers behind MAGSTIM are pioneers in the TMS industry. MAGSTIM is based in the United Kingdom. MAGSTIM is FDA cleared for depression treatment using the STIMGUIDE system. MAGSTIM also has FDA clearance for the iTBS, three minute (3 min.) protocol. The MAGSTIM HORIZON rTMS system is FDA cleared for three minute (3 min.), nineteen minute (19 min.), and thirty-seven-and-one-half minute (37.5 min.) protocols indicated for the treatment of major depressive disorder.

SOTERIX MEDICAL is based in the US. SOTERIX MEDICAL bundles the NEUROSOFT TMS system with its own neuro-navigation system that is known for not requiring line-of-sight. The SOTERIX MEDICAL system is capable of nineteen minute (19 min.), and thirty-seven-and-one-half minute (37.5 min.) treatments and can be used with or without neuro-navigation.

A MAGNUS NEUROMODULATION SYSTEM (MNS) with SAINT technology is a developing, rapid-acting brain stimulation technology designed to treat people suffering from major depressive disorder (MDD) who have not responded to existing treatments. The MAGNUS MEDICAL MODEL 1001k applies the MAGVENTURE rTMS and BRAIN SCIENCE tools/SOTERIX MEDICAL neuro-navigation.

Matryoshka dolls, more commonly known as Russian or babushka dolls, are a set of wooden figures nested into each other. This idea has also been employed in engineering and biology to describe something that is nested into each other a number of times. In the present disclosure, a hollow structure that nests within a smaller structure of similar shape, where such smaller structures may nest iteratively in even smaller structures of similar shape and so on, and also for structures being nested by bigger structures of similar shape is referred to as forming in an iteratively way, iterative construction, or some other like term.

With regard to the scientific principles that govern the present disclosure, one of skill in the art will recognize that molecules are affected by magnetic fields given that molecules are made of atoms. Such atoms are negatively charged if they comprise more negatively charged particles (e.g., electrons) than positively charged particles (e.g., protons). The opposite effect occurs, and the atom is positively charged if it contains fewer electrons than protons. So, particles or molecules can be categorized as positively charged (i.e., positive χ) or negatively charged (i.e., negative χ), where χ is their magnetic susceptibility. Their magnetization M can be expressed as

$$M = χ v . H \qquad ( 1 )$$

where
H is an applied magnetic field in emu/cm3,
M is magnetization of a particle after exposure to H, and
χ^{v} is a measured magnetic susceptibility of the molecules' electrons due to the magnetization.

Molecular substances can also be classified as polar or nonpolar. In a nonpolar molecule, the center of gravity of the positively charged nuclei and the electrons coincide, while in a polar molecule, they do not. In the absence of magnetic field, polar molecules are positioned randomly. Thus, their negative and positive charges are impossible to attach to each other, even though collisions between the molecules occur. However, when the samples are exposed to a magnetic field of certain intensity, the polar molecules are easily aligned in accordance with their positive and negative charges. Meanwhile, nonpolar molecules in the absence of a magnetic field move continuously at random because the positive and negative charges coincide in the centers of molecules. This inhibits coagulation. However, under the influence of a magnetic field, the positive and negative charges can be separated. The molecules are aligned in accordance with the direction of the magnetic field. With the resulting alignment, the molecules are in an orderly arrangement, causing the particles to coagulate and aggregate. In addition, the number of dipoles pointing in the direction of the field increases with increasing field strength. This makes it more likely that the particles coagulate and that uncommon or unnecessary particles or pollutants can be removed.

### Magnetic Gradient

The present inventor has recognized that some conventional theories assert that the effectiveness of a magnetic application ultimately depends not only on the magnetic strength but also on the magnetic gradient or magnetic flux concentration, which changes frequently along the magnetic device. The energy E produced by the magnetization M of a material and the magnetic field H for a volume V of the material can be expressed as follows:

$$E = - VM . H = - V ( χ v . H ) . H$$

This relationship can be simplified by assuming that the material load is parallel to the magnetic field and density when M is uniform. Thus, the magnetic interaction force F can be obtained as:

$$F = - ( dE / dx ) = ( χ v - χ o ) VH ( dH / dx )$$

where
χ^{o} is the magnetic susceptibility of the material that accommodates the magnetized material.

In calculation of the magnetic interaction force F, one important parameter that affects the effectiveness of magnetic applications is dH/dx, which indicates the rate of change of the magnetic field strength with distance, such ratio being referred to the magnetic gradient. When the magnetic field is uniform, dH/dx=0, so the particles are magnetized and aligned with the magnetic field. However, the particles are not exposed to a magnetic force that would ensure their separation from the solution. The magnetic gradient also becomes more significant when the volume V of material that is separated is small. The highest magnetic gradient is required in order to produce the strongest magnetic force on the particles for separation purposes. In addition, existence of the magnetic gradient and a magnetic field of alternating strength become more effective than a static magnetic field for the aggregation such as CaCO3. The implementation results in more rapid crystallization occurrences, thus enhancing the de-scaling process in a shorter period of time. Magnetic filtration can also be conducted by creating a magnetic gradient that can trap suspended solids and starch from a solution. In this case, the magnetic gradient can be developed by allowing a solution containing charged particles to flow through coils that are magnetized by permanent magnets located outside the pipes. The north and south poles of the magnets are arranged alternately in opposite directions to create an alternating magnetic field. A combination of coil magnetization and the magnetic field produces a magnetic gradient of higher intensity. This causes the flux lines to become considerably closer to each other and more concentrated. The flux intensity increases with increasing coil concentration. When the solution flows through the coils, the charged particles are attracted and thus separated from the original solution. In such embodiments, the intensity of the magnetic gradients can depend on the magnetic strength and the characteristics of the coil magnetization.

### Magnetic Memory

Magnetic memory can be defined as a period in which particles can sustain their magnetization properties after being exposed to a magnetic field of certain intensity. Magnetic memory phenomena have been reported, and the effects of magnetic memory on particles have been recorded over time periods ranging from ten (10) minutes to 150 hours. Some have found that the magnetic memory can be observed up to six (6) days after the exposure of a CaCO3 solution to magnetic field. In explaining magnetic memory, it has been postulated that when a magnetic field affects water molecules, the magnetic field changes the kinetic energy of the molecules. These occurrences change the momentum of the dipolar molecules and thus cause particles aggregation. The formed aggregates are stable and sufficiently large thereby making it difficult for them to return to their original shapes even after the magnets are removed. This indicates that the magnetic memory stored by the aggregates can last for a long time, possibly even permanently. Sometimes, The effects of magnetic memory on microorganisms or bacteria may differ from those on water molecules or other particles. For particles, higher magnetic memory is proven to improve the aggregation, but the condition is not so applicable for bacteria or microorganisms. Magnetic memory of either weak or high may improve or hinder their growth activity, thus influencing the performance of systems, especially the wastewater treatment system. Such consequences can be explained in terms of magnetic susceptibility. Different bacteria exist in a system and may have limitations on the susceptibility level towards the magnetic field. When the level is exceeded, the bacteria may die or exhibit growth reduction.

### Larmor Frequency

When a magnetic moment is placed in a magnetic field, the magnetic moment will tend to align with the field. Classically, a magnetic moment can be visualized as a current loop and the influence toward alignment can be described as the torque on the current loop exerted by the magnetic field. The idea of the magnetic moment as a current loop can be extended to describe the magnetic moments of orbital electrons, electron spins, and nuclear spins. In each case, the magnetic moment is associated with the angular momentum, and a torque can be identified, which tends to align the magnetic moment with the magnetic field. In the nuclear case, the angular momentum involved is the intrinsic angular momentum associated with the nuclear spin. When a magnetic moment is directed at some finite angle with respect to the magnetic field direction, the field will exert a torque on the magnetic moment. This torque causes the magnetic moment to precess about the magnetic field direction, which may be understood as analogous to the precession of a spinning top around the gravity field. The torque can be expressed as the rate of change of the nuclear spin angular momentum and equated to the expression for the magnetic torque on the magnetic moment, which when put in derivative form, gives a precession angular velocity.

The technology disclosed herein can also be visualized quantum mechanically in terms of the quantum energy of transition between the two possible spin states for spin ½. This can be expressed as a photon energy according to the Planck relationship. The magnetic potential energy difference is hυ=2 μB. The angular frequency associated with a “spin flip,” a resonant absorption or emission involving the spin quantum states is often written in the general form:

$$ω = gB$$

where
g is called the gyromagetic ratio (sometimes the magnetogyric ratio).

One of skill in the art will recognize that this frequency is a factor of two higher than the one earlier discussed because of the spin flip with energy change ΔE=2 μB. This nuclear spin transition for nuclei placed in a magnetic field is the basis for nuclear magnetic resonance (NMR). A useful, simplified version may be shown representing the Larmor frequency when B0=1; The gyromagnetic ratio (MHz/T) for a few commonly measured or imaged isotopes are:

(H-1:42.58, F-19:40.05, Na-23:11.26, P-31:17.24).

The near field and far field are regions of the electromagnetic field (EM) around an object, such as a transmitting antenna, are the result of radiation scattering off an object. Non-radiative “near-field” behaviors dominate close to the antenna or scattering object, while electromagnetic radiation ‘far-field’ behaviors dominate at greater distances. Far-field E (electric) and B (magnetic) field strength decreases as the distance from the source increases, resulting in an inverse-square law for the radiated power intensity of electromagnetic radiation. By contrast, near-field E and B strength decrease more rapidly with distance. The radiative field decreases by the inverse-distance squared, the reactive field by an inverse cubed law, thereby resulting in a diminished power in the parts of the electric field by an inverse fourth-power and sixth-power, respectively. The rapid drop in power contained in the near-field ensures that effects due to the near-field essentially vanish a few wavelengths away from the radiating part of the antenna.

The teaching of the present disclosure furthers the understanding of chemical changes in atoms and molecules under extreme conditions where magnetic forces counterbalance Coulombic forces. This is an area of fundamental chemistry research where, for instance, new phenomena are encountered such as perpendicular paramagnetic bonding. Further, the teaching of the present disclosure also advances that accurate data obtained using this methodology may help in the development of better functionals for the calculation of magnetic properties in density functional theory, a widely used method in computational chemistry. Referring back to Matryoshka dolls, embodiments of the present disclosure define a plurality of hollow structures that iteratively nest within smaller structures of similar shape.

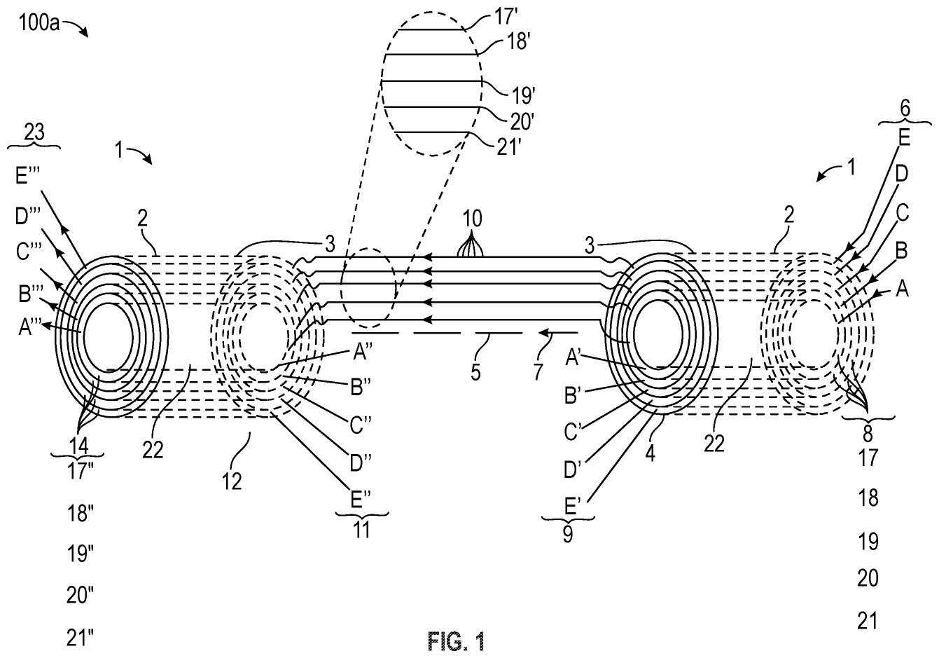

FIG. 1 is a novel neuromodulation embodiment 100a using arrays of iteratively shaped solenoids. The neuromodulation iteratively shaped solenoids array system 1 includes at least a plurality 2 of multiple tubular shaped solenoids 3, also known as iteratively shaped solenoids 3, that conform such multilayer arrays 4. Said tubular solenoids are aligned to each other along their longitudinal axis 5. The plurality 2 (two in the embodiment of FIG. 1) arrays 4 are formed by a number of such tubular shaped solenoids 3 with consecutively decremented diameter, placed one inside another. Each of the solenoids 3 is individually connected using separated input electrodes A, B, C, D, E 6 to send different electric signals. However, each said solenoid 3 may also be fed with the same electric parameters by electrically coupling all the input electrodes together producing a parallel connection of the solenoids 3 electric multilayer array 4. One of skill in the art will recognize that the direction of the electrical current through the wire loops is usually the same on all the electromagnets 3 of the same array 4. When the current runs through the solenoid's enameled wire on said multi-layer array 4, multiple magnetic fields are created concurrently. Given that most of the loops of the enameled wire run parallel to proximal enameled wire from the rest of the arrays 4, it produces an aligned multisource magnetic field that is not shown for clarity reasons, which subsequently synergizes the axial magnetic vector 7 and provides a longer magnetic field shape with less magnetic power loss over distance. Said multiple solenoids 3 are electrically connected in parallel with independent electric power sources, which of course can also be switched to a unique power source, by having a multilayer array 4 are characterized by having lower impedance than if the array only were a single solenoid with equivalent wire length. Accordingly, at the end of each emitter 8 solenoid 3, there are output electrodes A′, B′, C′, D′, E′ 9 that connect in at least one embodiment through cables 10 to their correspondent input electrode A″, B″, C″, D″, E″ 11 on the receiver 12 solenoids 3 array 4. So, in other words, the emitter 8 solenoids 17, 18, 19, 20, 21 connect through separated coaxial 10 cables 17′, 18′, 19′, 20′, 21′ with the receiver 14 solenoids 17″, 18″, 19″, 20″, 21″. Of course, the individual connections of each emitter 8 solenoids 3 may be switched in order to be all connected to a single coaxial cable that connects on its other end to all the input electrodes A″, B″, C″, D″, E″ on the receiver 12 multi solenoid 3 array. Additionally, in some embodiments, each array's core 22 may be made of iron, permalloy (nickel-iron), as well as an alloy known as Mu-Metal, each one with high magnetic permittivity in order to increase inductance. Finally, electric energy emerges through output electrodes A′″, B′″, C′″, D′″, E′″ 23. The embodiment of FIG. 1 includes features that better produce a more concentrated and longer magnetic field crossing from the emitter 8 to the receiver 14 solenoids that optimize magnetic power transfer.

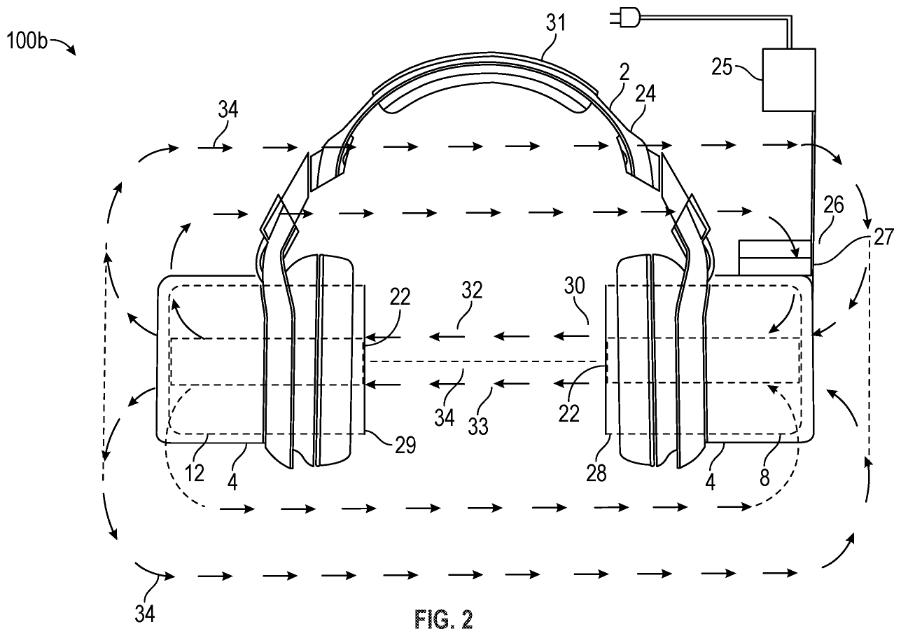

FIG. 2 is a transcranial magnetic stimulation device 100b that employs an array of coupled iteratively shaped solenoids. In the embodiment of FIG. 2, a neuromodulation iteratively shaped solenoids coupled array system 1 is held by a diadem shaped holder device 24 that optionally is arranged as a set of headphones. This device 2 includes a pair of iteratively shaped solenoids arrays 4 where the emitter 8 solenoids 4 receives the electric power from the power source 25 where in at least one embodiment, said power source is a pluggable one, transforming alternate current of 60 Hz or some other frequency into one (1) Ampere ridged electric signal. Before said electric power is really received by the emitter solenoids 8 solenoids array 4, the control of the device's electric circuits is primarily fed by said power source. Said controls are comprised by an electronic control activation circuits subsystem 26 and a therapy parameters electronic control 27 circuits subsystem. The first subsystem 26 is designed to manage each treatment by accounting for the length and number of therapeutic sessions provided and to restart the counter for another treatment (i.e., to ‘refill’ the device 2). The second subsystem 27 allows the user to manipulate within safe parameters, the frequency (Hz) and length (e.g., milliseconds, seconds, or some other time period) of signal as well as the magnetic power (e.g., Teslas) of the magnetic field that goes from the emitter array 28 to the receiver 29 array. So, once the electric power reaches the emitter multi layered tubular solenoids array 28, it is divided amongst each solenoid's wire loops.

As explained herein, while each electron runs along the loops of said tubular solenoids, multiple parallel simultaneous magnetic fields are created, thereby facilitating a multi emitter magnetic field 30. Given that the loops are parallel and the direction of the current is very much parallel through each solenoid in proximity, the concurrently emitted magnetic fields present the tendency to also be parallel and to accumulate. One of skill in the art will recognize that in some embodiments, the electricity coming out from each emitter solenoid 8 is collected by an axial cable 31 in order to reach the receiver 29 iteratively shaped solenoids 8 array where, again, the electric power is coupled to each solenoid in an electrically parallel connection. Such embodiments also produce multiple simultaneous magnetic fields that are directly electrically fed from the emitter 28 array.

One of skill in the art will recognize that the resultant magnetic field 32 of the emitter 28 array is coupled with the resultant magnetic field of the receiver 29 array. This kind of magnetic field coupling that uses the electric and magnetic resultants from an emitter 28 array into another similarly shaped receiver 29 array enables magnetic power transfer at longer distances, and this technology also works even if the feeding current is direct current (DC) revealing this effect to be similar but different from the resonant magnetic effect. One of skill in the art will recognize that the embodiment of FIG. 2 shows that the magnetic field generated between emitter 28 array and receiver 29 array is capable of sending such magnetic power through the human cranium to cross through the brain. Different from standard TMS embodiments, the magnetic lines of force 33 rise from the core 22 of the emitter 28 array and run through a straight and concentrated trajectory into the core 22 of the receiver 29 array. This feature provides several advantages including more accurate aiming since all other transcranial magnetic stimulation devices provide curved and sparse trajectories. Having this in mind, this particular straight and concentrated trajectory of core to core magnetic field force 33 lines become extremely helpful in order to achieve neural modulation to specific selected regions of the brain, and this advantage is even more appreciated when said selected regions of the brain are deeply located. Therefore, despite other neuromodulation systems, embodiments of an iteratively shaped solenoids array system 1 as disclosed herein are operative simply by placing the working emitter array at one side of the cranium and the working receiver array on the contralateral side of said cranium to make said magnetic power to cross, side to side, through the brain, in a concentrated and straight beam of magnetic energy.

The straight-line embodiments of the present disclosure can achieve the crossing of said magnetic energy beam anywhere through the skull and brain. However, as an expert on neurologic modulation may say, a specific anatomic target should be selected to achieve a more specific therapeutic response. One of skill in the art will recognize, however, that what has been achieved so far with all conventional types of transcranial magnetic stimulation is to sparsely stimulate neurotransmitter release, mostly on the sulci, given that such conventional devices are more perpendicularly oriented to the elliptical force trajectory of the intruding transcranial standard magnetic field. In more detail, one of skill in the art will recognize that the cerebral cortex is the outer grey matter layer that completely covers the surface of the two cerebral hemispheres. It is about 2 to 4 mm thick and contains an aggregation of nerve cell bodies. This layer is thrown into complex folds, with elevations called gyri and grooves known as sulci.

The embodiment of FIG. 2 identifies placement of a common axis 34, and consequently, main magnetic force lines 33 between the emitter 28 and receiver 29 arrays may be through any trajectory across the human skull by simply placing the skull between said emitter 28 and receiver 29 arrays at any desired position. So, the placement of said arrays with their common axis aimed to interact with anatomical targets within the brain is now possible because such magnetic concentrated straight magnetic power is capable to reach the supraorbital cortex bilaterally and or the Accumbens Nuclei, since both are located on the anterior region of the brain. One of skill in the art will recognize that any other neurologic structure is also within reach of said magnetic field by simply moving and aiming the system 1. Advantageously, certain vulnerable areas of the anatomy may be avoided by such precision aiming, and target structures in close proximity to encephalic regions, such as the brain stalk, may now, with this advancement, also be reached and stimulated since the main electric power is not too dispersed or traveling through elliptical unpredictable trajectories as it is the case with other magnetic technologies.

Another observation is that, as shown in the embodiment of FIG. 2, in any magnetic system, the magnetic power that travels concentrated 33 from core 22 to core 22, once emerging from the receiver array's core, it is dispersed within a much bigger tubular shaped returning area 34 from the receiver 29 back to the emitter 28, exposing the neural tissue present to a much less concentrated magnetic power. The neuromodulation device of FIG. 2 employs a more concentrated and longer magnetic field 33. The shape of the signals sent through such magnetic field from emitter 28 to receiver 29 arrays, crossing through the skull, depends on the parameters of the electrical signal source (e.g., current, voltage, frequency, waveform, duty cycle, synchronicity of the electric signals, and the like) having in mind that each emitter 28 solenoid may be electrically fed with independent electrical signals (e.g., FIG. 1), therefore creating a multiplicity of electrical stimulus on the brain. Each solenoid of the 29 receiver array may also be electrically coupled independently, therefore obtaining a multiplicity of electrical responses directly coming from the brain. In such embodiments, a neural link may be established where multiple signaling is delivered concurrently at different points within the boundaries of said synchronic magnetic field by internal signal discrepancies within said magnetic field. So, given that multiple signals at different points within a region are magnetic inputs into the brain, multiple responses to such stimulus can be elicited and received as an output from the brain. Along these lines, one of skill in the art will recognize that the iterative shaped solenoids array with parallel walls can be arranged to produce multiple, concurrent magnetic fields, and since they travel on the same direction, they accumulate and create a bigger magnetic resonant field due to the far field magnetic power transfer effect, and the uses of such effect during neuromodulation provide never-before-seen applications in neural transduction and biochemical catalysis. Additional data survey of electrical transducers from the brain (e.g., electro-encephalogram and related technologies including artificial intelligence) may be used to enhance the transcription and interaction of this novel magnetic technology.

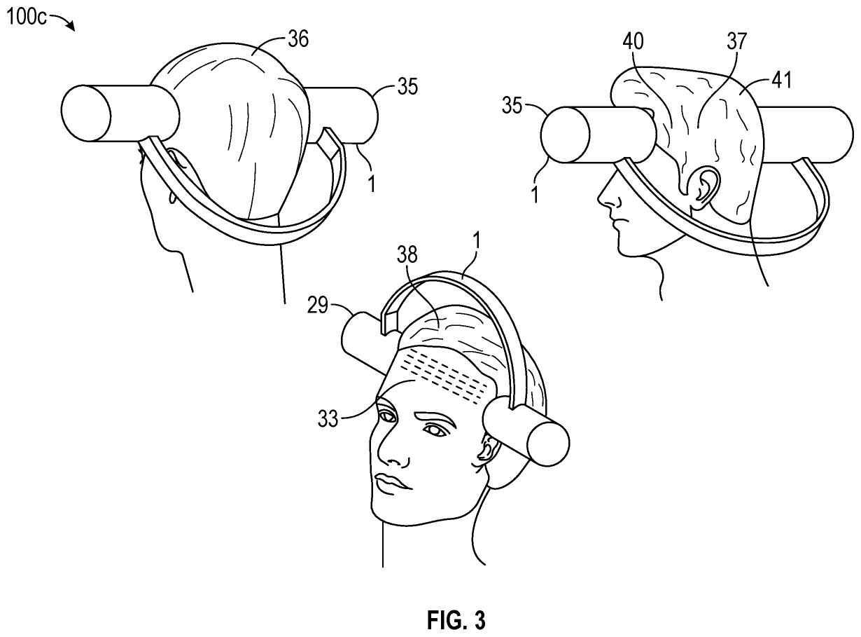

FIG. 3 is an intracranial electromagnetic array 100c embodiment for intra parenchyma, endovascular, and endocavity neuromodulation magnetic transduction. The embodiment of FIG. 3 includes neuromodulation iteratively shaped solenoids coupled array system 1 and illustrates how data transfer may be exchanged and refined in order to achieve an optimized neural transducer 35 patient (e.g., human, non-human) thinking-artificial intelligence interaction. The embodiment of FIG. 3 includes multiple coupled arrays 1 that may be placed at different positions in order to interact with specific specialized brain regions. One of skill in the art will recognize that the magnetic field may, for example, be placed crossing the occipital 36 brain cortex in order to interact with the visual processing system. Additionally, or alternatively, the magnetic field may be arranged to cross the left lateral temporal 37 cortex in order to interact with the auditory processing system. As explained with respect to FIG. 2, such interaction starts with magnetic stimulation of the neural area of interest using variables such as frequency, magnetic power, electrical signal shape, and the like to produce variations on the multiple emitted magnetic field 33. Based on the available harmonics that may be elicited by the multiple types of signals and combination of simultaneous individual solenoid activation in a similar way, a philharmonic orchestra sends simultaneously a great quantity of different, multi-frequency, harmonic related notes, and these different stimulations elicit multiple electric responses from the neural systems of brains that receive the electrical representations of the auditory signals. Such information is recollected individually by each receiving 29 solenoid 8 and may also be recollected by electroencephalographic means as well as direct interaction with the patient's own neural modulation. Such recollected information is then analyzed using artificial intelligence in order to learn a communication pattern. One of skill in the art will recognize that such interaction may occur in an input fashion while multiple magnetic signals are affecting neural circuits and in an output fashion while the response of the brain is recollected in the shape of clinical signs and symptoms; the change of the magnetic field recollected by the receiver solenoids as well as the electric changes recollected by the electroencephalogram.

As shown in FIG. 3, when the magnetic field goes through the frontal region 38, the system desirably interacts with the emotion-managing area of the brain. When the magnetic field 33 goes through the occipital region 36, the image-forming region of the brain is stimulated. When the magnetic field 33 goes through the high temporal 40 and low parietal 41 regions, the spoken language area of the brain is stimulated. So, depending on where the magnetic field 33 produces stimulation, different responses are expected to be sensed, recorded, studied, and understood. So, as discussed herein, such subtle electromagnetic changes happening within the brain and also within the crossing magnetic field 33 boundaries can be sensed by the receiving 28 multilayer solenoids arrays in order to be understood as output data.

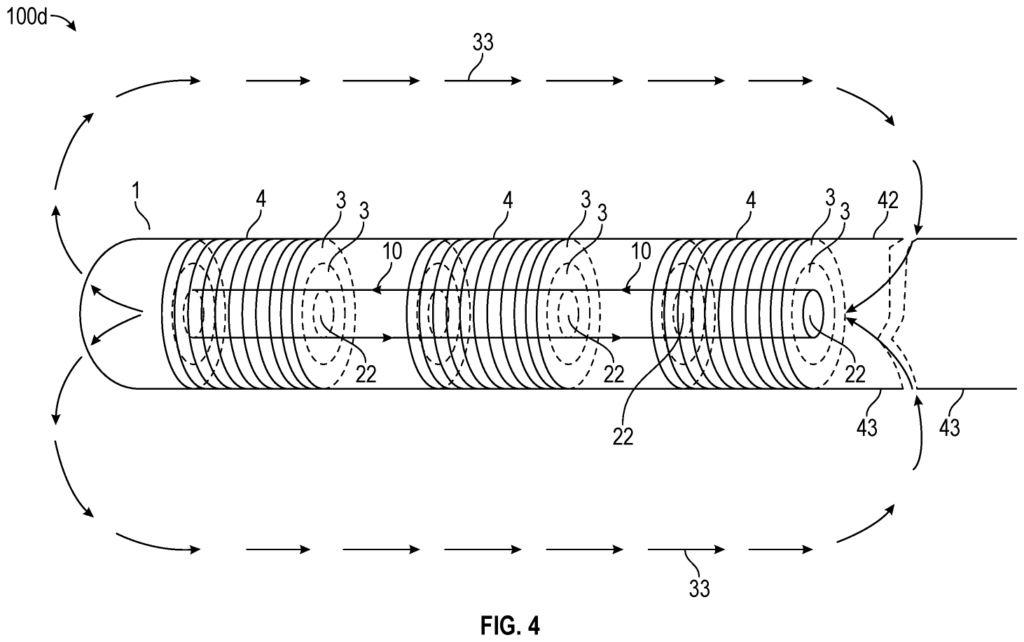

FIG. 4 is a transcranial magnetic stimulation device 100d embodiment that employs far field technology. The embodiment of FIG. 4 includes a miniaturized neuromodulation iteratively shaped solenoids coupled array system 1 that can be directly inserted into the brain or a vessel as a single or paired system. This embodiment includes a miniaturized neuromodulation iteratively shaped solenoids coupled array system 1 located on the tip 42 of a catheter 43. Said catheter 43 may reach the endo-cranial space by direct insertion of such catheter into the brain's parenchyma, or it may also reach inside the cranium by traveling through a blood vessel. Other ways are also contemplated. Whatever way the cranial cavity is reached, this device 100d enables a low inductance, coupled emitter-receiver (i.e., emitter/receiver) system where the main axis of the magnetic field 33 runs inside the catheter 43, but the external diameter of such magnetic field 33 delivers a better distributed electromagnetic power than simple electrode-based technology (i.e., electric field) based catheters 43 for deep brain stimulation. With multiple, iterative solenoid 8 arrays along such catheter 42, a longer and wider neural modulation magnetic field 33 volume may be created. As explained herein, the electrical feeding of the iterative solenoids in at least some embodiments is connected in parallel and the connection between arrays is serial.

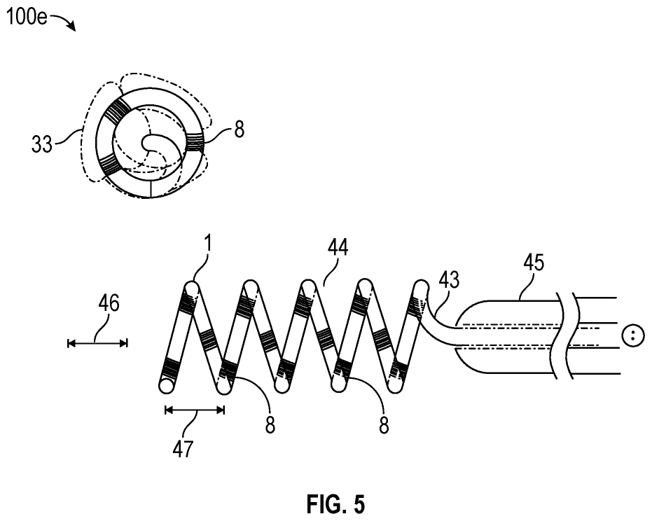

FIG. 5 is another intracranial electromagnetic array 100e embodiment for intra parenchyma, endovascular, and endocavity neuromodulation magnetic transduction. The embodiment of FIG. 5 includes a miniaturized neuromodulation iteratively shaped solenoids coupled array system 1 that can also be directly inserted into the brain. In this case, the electrode system may be electric and helicoidally shaped 44 and may also include iterative solenoid 8 arrays within such helicoidally shaped catheter 43 to achieve a bigger magnetic field volume of interaction with the neuronal circuits within the brain. The technique of insertion may be the same as of a parenchymal catheter. Some differences may be that this catheter 43 emerges through a rigid straight tubular carrier 45 of slim walls, which stops at a pre-deployment depth, and the helicoid 44 catheter goes from a straight shape 46 forced by the rigid wall of such tubular carrier 45 into recovering its original helicoid shape 44 thanks to its helicoidally shaped memory elastic proprieties after emerging from said rigid tubular carrier 45. One of skill in the art will recognize that to reduce a likelihood or amount of brain damage during the insertion of a helicoidally shaped catheter 43 in some embodiments, the depth of advance 46 every 360 degrees turn may be arranged as equal to its axial length 47 between turns. This embodiment of a miniaturized neuromodulation iteratively shaped solenoids coupled array system 1 has a helicoidally shaped axis 5 where the magnetic field 33 created becomes wider, and the bends 48 of the central magnetic flux may also interact outside the tip of the catheter.

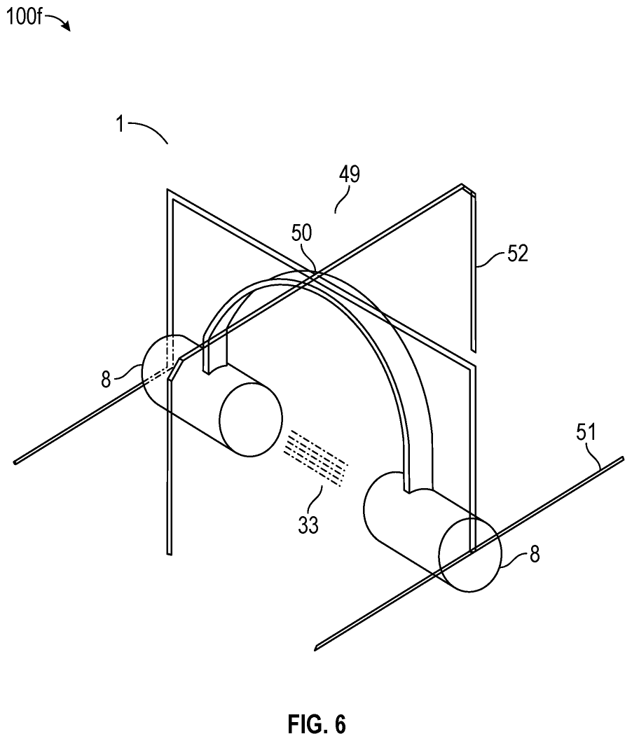

FIG. 6 is an array 100f embodiment of neuromodulation iteratively shaped solenoids where a multisource magnetic field is employed concurrently with electromagnetic radiation on the frequency range of radio and microwaves to produce atomic and molecular catalysis. In the embodiment of FIG. 6, a neuromodulation iteratively shaped solenoids coupled array system 1 includes an advanced electromagnetic catalysis system 49. The catalysis system 49 includes at least two multi-coil 8 arrays, where said plurality (e.g., pair) of coils systems include iteratively shaped solenoids arrays 1 that produce a strong magnetic field created by the interaction of coupled pairs of iterative shaped multi-solenoid arrays. The strong magnetic field B0 5 of approximately 0.5 to 35 Tesla, or more when possible, may affect the orientation of the spin axis of protons of the target molecules within the matter that is located within the borders of such strong magnetic field 33 along the X axis. There is a second system of coils that includes an array of emitter/receiver antennas 50 that produce far-field electromagnetic radiation from different perpendicular emission angles Y 51, Z 52 as emitter antennas (i.e., radiation sources) and in a synchronized manner also target molecules located within the matter located inside of such magnetic field 33.

The specific way the innovation of the present disclosure alters matter includes the torsion and rotation of the molecules. The torsion created by electromagnetic energy on the microwave and/or radio frequencies affecting the molecules is given as a function of its dipolar charge and also produces heat. However, this present innovation is implemented by change in orientation of the spin axis of protons provided by the magnetic B0 plus the far-field electromagnetic radiation B1 that produces molecular rotation and torsion, enhancing molecular instability. The molecular increased instability is produced by the synergic effect of a strong magnetic field and radio frequency at a specific atomic or molecular Larmor's frequency.

In some cases, according to the kind of chemical reaction expected, an electromagnetic cage is also present as a part of the transverse electromagnetic resonator. The cage 30 is arranged to bounce back the electromagnetic radiation from the walls of the cage 30 millions of times through the target matter in a similar way as what happens inside of a microwave oven. A similar but not equal effect is achieved by the magnetic resonance imaging (MRI) apparatus with a difference that the MRI has the Larmor's resonant frequency tuned to the hydrogen atoms of the water molecules (H₂O). The MRI only affects the spinning angle of the electrons by providing extra magnetic energy; generating photonic radiation when the electrons return to a lower energy level. The near-field photonic radiation is sensed by the transverse electromagnetic (TEM) receiving antennas, generating images accordingly to water and hydrogen variation amongst tissues. The catalysis system of the present disclosure has the advantage over MRI because molecules other than water and atoms other than Hydrogen may now be targeted in order to produce a resonant effect. Another advantage over the standard MRI lies in the fact that changes on B0 5 intensity produces a change on Larmor's resonant frequency. Thus, different Larmor's frequencies from same targets may be elicited accordingly to which frequency best produces molecular instability, being the targeting far-field electromagnetic radiation frequencies emitted by the emitter antennas of the variable field B1. So, the difference with the instability that produces heat, where heat affects all present molecules, relies on the so-called magneto-radiation resonance catalysis system and method.

Such magneto-radiation resonance catalysis effect is based on the fact that specific atoms and molecules may be targeted (i.e., using Larmor's frequencies) without overheating the targeted matter. Customized instability may now be particularly enhanced accordingly to the Larmor's resonant frequency of each chosen chemical element and/or molecule, particularly when the molecules are also being stimulated by added up resonant far-field electromagnetic waves. The resonant electromagnetic effect also produces large electron jumps on matter that have not been yet well understood on modern physics. Given that specific molecules may now be forced to become more instable, the selective molecular targeting for catalysis on chemistry and biochemistry is an advantage taught in the present disclosure. As an example of expected applications of this novel technology, without departing from the scope of the innovation, a viral spike (e.g., one from a corona virus such as COVID-19) is made of nucleotides and sugars (i.e., glucose) that the virus employs to recognize and attach to cellular animal (e.g., human, non-human) membranes. It is known that the spike is fragile and becomes unstable and breaks down under laboratory observation, for instance, at 40 degrees Celsius and with a pH of 8. Thus, magneto-radiation catalysis by the targeting of the spikes or other molecules of other infectious agents is expected.

In order to change the Larmor's frequency when desired, and given that the frequency changes under a variable strong magnetic field, the magneto-radiation catalysis may also be performed under the effects of a fluctuating strong magnetic field (Bo variable). Given that the magneto-radiation catalysis teaching disclosed herein enhances chemical reactions, reactants in the form of chemical compounds may and should also be present for better results. Note that given that this novel technology provides the possibility of interaction with complex proteins by catalyzing specific types and sub-types of biochemical compounds, it may be possible to produce even genetic therapy. For instance, if histones (i.e., proteins that repair DNA) are catalyzed and are made more prone to be catalyzed (i.e., made more prone for the chemical reaction the molecule performs itself or it might also facilitate, behaving as an enzyme), the diseases related to imperfect genetic expression might now be treated using this kind of DNA repair (e.g., cancer, Alzheimer's disease, Aging, and the like). One of skill in the art will recognize that according to at least one embodiment, it is possible to use the Larmor's resonant frequencies of different elements to achieve desired reactions. One of the applications is to retrieve calcium and lipids from the lumen of an artery. For that matter, an animal (e.g., human, non-human) may be placed under the influence of the strong magnetic field, and the electromagnetic radiation on the radio to microwave frequencies spectrum, using the Larmor's resonant frequency of calcium (instead of hydrogen) to enhance calcium release from the inner wall of the patient's arteries. One of skill in the art will recognize that during the catalysis of the desired molecules, additional energy such as ultrasound or laser may be also applied to the desired target matter in order to better remove calcium from the inner wall. Some atoms are more prone to be affected by electromagnetic radiation according to their atomic valence.

Having now set forth certain embodiments, further clarification of certain terms used herein may be helpful to providing a more complete understanding of that which is considered inventive in the present disclosure.

In the embodiments of present disclosure, one or more particular components and devices of the embodiments are interchangeably described herein as “coupled,” “connected,” “attached,” and the like. It is recognized that once assembled, the system is suitably connected, sealed, and otherwise formed to a mechanically, medically, or otherwise industrially acceptable level.

The figures in the present disclosure lend themselves to one or more non-limiting computing device embodiments that control the operation and parameters of electromagnets. The computing devices may include operative hardware found in conventional computing device apparatuses such as one or more processors, volatile and non-volatile memory, serial and parallel input/output (I/O) circuitry compliant with various standards and protocols, wired and/or wireless networking circuitry (e.g., a communications transceiver), one or more user interface (UI) modules, logic, and other electronic circuitry.

To the extent that such computing devices include processing devices, or “processors,” these processors include central processing units (CPU's), microcontrollers (MCU), digital signal processors (DSP), application specific integrated circuits (ASIC), peripheral interface controllers (PIC), state machines, and the like. Accordingly, a processor as described herein includes any device, system, or part thereof that controls at least one operation, and such a device may be implemented in hardware, firmware, or software, or some combination of at least two of the same. The functionality associated with any particular processor may be centralized or distributed, whether locally or remotely. Processors may interchangeably refer to any type of electronic control circuitry configured to execute programmed software instructions. The programmed instructions may be high-level software instructions, compiled software instructions, assembly-language software instructions, object code, binary code, micro-code, or the like. The programmed instructions may reside in internal or external memory or may be hard-coded as a state machine or set of control signals. According to methods and devices referenced herein, one or more embodiments describe software executable by the processor, which when executed, carries out one or more of the method acts.

The embodiments of the present disclosure lend themselves to include or otherwise cooperate with one or more computing devices. It is recognized that these computing devices are arranged to perform one or more algorithms to implement various concepts taught herein. Each of said algorithms is understood to be a finite sequence of steps for solving a logical or mathematical problem or performing a task. Any or all of the algorithms taught in the present disclosure may be demonstrated by formulas, flow charts, data flow diagrams, narratives in the specification, and other such means as evident in the present disclosure. Along these lines, the structures to carry out the algorithms disclosed herein include at least one processing device executing at least one software instruction retrieved from at least one memory device. The structures may, as the case may be, further include suitable input circuits known to one of skill in the art (e.g., keyboards, buttons, memory devices, communication circuits, touch screen inputs, and any other integrated and peripheral circuit inputs (e.g., accelerometers, thermometers, light detection circuits and other such sensors)), suitable output circuits known to one of skill in the art (e.g., displays, light sources, audio devices, tactile devices, control signals, switches, relays, and the like), and any additional circuits or other structures taught in the present disclosure. To this end, every invocation of means or step plus function elements in any of the claims, if so desired, will be expressly recited.

As known by one skilled in the art, a computing device has one or more memories, and each memory comprises any combination of volatile and non-volatile computer-readable media for reading and writing. Volatile computer-readable media includes, for example, random access memory (RAM). Non-volatile computer-readable media includes, for example, read only memory (ROM), magnetic media such as a hard-disk, an optical disk, a flash memory device, a CD-ROM, and/or the like. In some cases, a particular memory is separated virtually or physically into separate areas, such as a first memory, a second memory, a third memory, etc. In these cases, it is understood that the different divisions of memory may be in different devices or embodied in a single memory. The memory in some cases is a non-transitory computer medium configured to store software instructions arranged to be executed by a processor. Some or all of the stored contents of a memory may include software instructions executable by a processing device to carry out one or more particular acts.

The computing devices described herein may further include operative software found in a conventional computing device such as an operating system or task loop, software drivers to direct operations through I/O circuitry, networking circuitry, and other peripheral component circuitry. In addition, the computing devices may include operative application software such as network software for communicating with other computing devices, database software for building and maintaining databases, and task management software where appropriate for distributing the communication and/or operational workload amongst various processors. In some cases, the computing device is a single hardware machine having at least some of the hardware and software listed herein, and in other cases, the computing device is a networked collection of hardware and software machines working together in a server farm to execute the functions of one or more embodiments described herein. Some aspects of the conventional hardware and software of the computing device are not shown in the figures for simplicity.

Amongst other things, exemplary computing devices of the present disclosure may be configured in any type of mobile or stationary computing device such as a remote cloud computer, a computing server, a smartphone, a tablet, a laptop computer, a wearable device (e.g., eyeglasses, jacket, shirt, pants, socks, shoes, other clothing, hat, helmet, other headwear, wristwatch, bracelet, pendant, other jewelry), vehicle-mounted device (e.g., train, plane, helicopter, unmanned aerial vehicle, unmanned underwater vehicle, unmanned land-based vehicle, automobile, motorcycle, bicycle, scooter, hover-board, other personal or commercial transportation device), industrial device (e.g., factory robotic device, home-use robotic device, retail robotic device, office-environment robotic device), or the like. Accordingly, the computing devices include other components and circuitry that is not illustrated, such as, for example, a display, a network interface, memory, one or more central processors, camera interfaces, audio interfaces, and other input/output interfaces. In some cases, the exemplary computing devices may also be configured in a different type of low-power device such as a mounted video camera, an Internet-of-Things (IoT) device, a multimedia device, a motion detection device, an intruder detection device, a security device, a crowd monitoring device, or some other device.

When so arranged as described herein, each computing device may be transformed from a generic and unspecific computing device to a combination device arranged comprising hardware and software configured for a specific and particular purpose such as to provide a determined technical solution. When so arranged as described herein, to the extent that any of the inventive concepts described herein are found by a body of competent adjudication to be subsumed in an abstract idea, the ordered combination of elements and limitations are expressly presented to provide a requisite inventive concept by transforming the abstract idea into a tangible and concrete practical application of that abstract idea.

Software may include a fully executable software program, a simple configuration data file, a link to additional directions, or any combination of known software types. When a computing device updates software, the update may be small or large. For example, in some cases, a computing device downloads a small configuration data file to as part of a software update, and in other cases, a computing device completely replaces most or all of the present software on itself or another computing device with a fresh version. In some cases, software, data, or software and data is encrypted, encoded, and/or otherwise compressed for reasons that include security, privacy, data transfer speed, data cost, or the like.

Database structures, if any are present in the systems described herein, may be formed in a single database or multiple databases. In some cases, hardware or software storage repositories are shared amongst various functions of the particular system or systems to which they are associated. A database may be formed as part of a local system or local area network. Alternatively, or in addition, a database may be formed remotely, such as within a distributed “cloud” computing system, which would be accessible via a wide area network or some other network.

Input/output (I/O) circuitry and user interface (UI) modules include serial ports, parallel ports, universal serial bus (USB) ports, IEEE 802.11 transceivers and other transceivers compliant with protocols administered by one or more standard-setting bodies, displays, projectors, printers, keyboards, computer mice, microphones, micro-electro-mechanical (MEMS) devices such as accelerometers, and the like.

In at least one embodiment, devices as discussed herein may communicate with other devices via communication over a network. The network may involve an Internet connection or some other type of local area network (LAN) or wide area network (WAN). Non-limiting examples of structures that enable or form parts of a network include, but are not limited to, an Ethernet, twisted pair Ethernet, digital subscriber loop (DSL) devices, wireless LAN, Wi-Fi, Worldwide Interoperability for Microwave Access (WiMax), or the like.

In the present disclosure, memory may be used in one configuration or another. The memory may be configured to store data. In the alternative or in addition, the memory may be a non-transitory computer readable medium (CRM). The CRM is configured to store computing instructions executable by a processor. The computing instructions may be stored individually or as groups of instructions in files. The files may include functions, services, libraries, and the like. The files may include one or more computer programs or may be part of a larger computer program. Alternatively, or in addition, each file may include data or other computational support material useful to carry out the computing functions of a system described in the present disclosure.

Buttons, keypads, computer mice, memory cards, serial ports, bio-sensor readers, touch screens, and the like may individually or in cooperation be useful to a user operating the far field magnetic power transfer (FFMPT) devices described herein. The devices may, for example, input control information into the system. Displays, printers, memory cards, LED indicators, temperature sensors, audio devices (e.g., speakers, piezo device, etc.), vibrators, and the like are all useful to present output information to the user operating the described systems. In some cases, the input and output devices are directly coupled to the FFMPT system and electronically coupled to a processor or other operative circuitry. In other cases, the input and output devices pass information via one or more communication ports (e.g., RS-232, RS-485, infrared, USB, etc.).

As described herein, for simplicity, a medical practitioner or other use may in some cases be described in the context of the male gender. It is understood that a medical practitioner or other user can be of any gender, and the terms “he,” “his,” and the like as used herein are to be interpreted broadly inclusive of all known gender definitions. As the context may require in this disclosure, except as the context may dictate otherwise, the singular shall mean the plural and vice versa; all pronouns shall mean and include the person, entity, firm or corporation to which they relate; and the masculine shall mean the feminine and vice versa.

In the absence of any specific clarification related to its express use in a particular context, where the terms “substantial” or “about” in any grammatical form are used as modifiers in the present disclosure and any appended claims (e.g., to modify a structure, a dimension, a measurement, or some other characteristic), it is understood that the characteristic may vary by up to 30 percent. For example, a first electromagnetic array may be described as being formed or otherwise oriented “substantially along a common axis.” In these cases, an axis that is oriented exactly vertical is oriented along a “Z” axis that is normal (i.e., 90 degrees or at right angle) to a plane formed by an “X” axis and a “Y” axis. Different from the exact precision of the term, “vertical,” the use of “substantially” to modify the characteristic permits a variance of the “vertical” characteristic by up to 30 percent. Accordingly, an axis that is oriented “substantially vertical” includes axes oriented between 63 degrees and 117 degrees. An axis that is oriented at 45 degrees of an X-Y plane, however, is not oriented “substantially vertical.” As another example, a solenoid having a particular linear dimension of “between about three (3) inches and five (5) inches” includes such devices in which the linear dimension varies by up to 30 percent, Accordingly, the particular linear dimension of the solenoid may be between one point five (1.5) inches and six point five (6.5) inches.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, the technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

In the present disclosure, when an element (e.g., component, circuit, device, apparatus, structure, layer, material, or the like) is referred to as being “on,” “coupled to,” or “connected to” another element, the elements can be directly on, directly coupled to, or directly connected to each other, or intervening elements may be present. In contrast, when an element is referred to as being “directly on,” “directly coupled to,” or “directly connected to” another element, there are no intervening elements present.

The terms “include” and “comprise,” as well as derivatives and variations thereof, in all of their syntactic contexts, are to be construed without limitation in an open, inclusive sense, (e.g., “including, but not limited to”). The term “or,” is inclusive, meaning and/or. The phrases “associated with” and “associated therewith,” as well as derivatives thereof, can be understood as meaning to include, be included within, interconnect with, contain, be contained within, connect to or with, couple to or with, be communicable with, cooperate with, interleave, juxtapose, be proximate to, be bound to or with, have, have a property of, or the like.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In the present disclosure, the terms first, second, etc., may be used to describe various elements, however, these elements are not to be limited by these terms unless the context clearly requires such limitation. These terms are only used to distinguish one element from another. For example, a first machine could be termed a second machine, and, similarly, a second machine could be termed a first machine, without departing from the scope of the inventive concept.

The singular forms of “a,” “an,” and “the” in the present disclosure include plural referents unless the content and context clearly dictates otherwise. The conjunctive terms, “and” and “or,” are generally employed in the broadest sense to include “and/or” unless the content and context clearly dictates inclusivity or exclusivity as the case may be. The composition of “and” and “or” when recited herein as “and/or” encompasses an embodiment that includes all of the elements associated thereto and at least one more alternative embodiment that includes fewer than all of the elements associated thereto.

In the present disclosure, conjunctive lists make use of a comma, which may be known as an Oxford comma, a Harvard comma, a serial comma, or another like term. Such lists are intended to connect words, clauses, or sentences such that the thing following the comma is also included in the list.

The headings and Abstract of the Disclosure provided herein are for convenience only and do not interpret the scope or meaning of the embodiments.

The various embodiments described above can be combined to provide further embodiments. Various features of the embodiments are optional, and features of one embodiment may be suitably combined with other embodiments. Aspects of the embodiments can be modified, if necessary, to employ concepts of the various patents, application and publications to provide yet further embodiments.

Example 1 is neuromodulation device that employs far-field magnetic transfer effect, and it includes a plurality (i.e., at least a pair) of iterative multiple-magnet arrays where each array includes a plurality of magnets iteratively (e.g., consecutively nested) into a bigger one with substantially parallel walls in a multilayered fashion to better produce said effect.

Example 2 is an example in concordance with Example 1 or with Example 1 and any one or more other Examples disclosed herein where it is the magnetic force that emerges from the emitter core of a multi coil array, the one employed for neuromodulation and neurotransduction.

Example 3 is an example in concordance with Example 1 or with Example 1 and any one or more other Examples disclosed herein where on the application of the magnetic power is emitted to disrupt the electric signaling of a neural circuit such as the Supra-Orbital cortex with the Accumbens Nucleus circuit.

Example 4 is an example in concordance with Example 1 or with Example 1 and any one or more other Examples disclosed herein where the application of the magnetic power is emitted to stimulate the electric signaling of a neural circuit as the prefrontal cortex with the subthalamic nucleus circuits.

Example 5 is a neuromodulation technique in concordance with Example 1 or with Example 1 and any one or more other Examples disclosed herein where a catheter inserted on the brain deploys a helicoidally shaped coil that emerges from its distal end and it is introduced through parenchyma by rotational motion.

Example 6 is an example in concordance with Example 1 or with Example 1 and any one or more other Examples disclosed herein where such helicoidally shaped coil includes a miniature multi coil array to generate a resonant magnetic field.

Example 7 is an example in concordance with Example 1 or with Example 1 and any one or more other Examples disclosed herein where the emitter array and the receiver arrays are electrically coupled in serial and the solenoids of each array are electrically coupled in parallel to control the device parameters.

Example 8 is an example in concordance with Example 1 or with Example 1 and any one or more other Examples disclosed herein where each emitter solenoid is electrically fed independently to send multiple signal variations to said solenoids, consequently producing a complex multi-emitter magnetic field to generate a complex data input transducer capable of interacting with a much bigger neuronal population in a concentrated region of the brain.

Example 9 is an example in concordance with Example 1 or with Example 1 and any one or more other Examples disclosed herein where each receiver solenoid is electrically independently coupled to retrieve the signal variations collected by each solenoid of such receiver array, and to generate a complex data output transducer capable of sensing a much bigger neuronal population in a concentrated region of the brain.

Example 10 is an example in concordance with Example 1 or with Example 1 and any one or more other Examples disclosed herein where a catheter inserted on the brain includes at least two miniature multi-solenoids arrays arranged to produce a multi-emitter magnetic field in a particular region of the brain.

Example 11 is an example in concordance with Example 1 or with Example 1 and any one or more other Examples disclosed herein where a catheter inserted in the brain includes at least two miniature multi-solenoids arrays and is arranged to deploy a helicoidally shaped coil that emerges from its distal end, said device introduced through parenchyma by rotational motion where such catheter contains at least two multi solenoid arrays arranged to produce a multi emitter magnetic field in a wider and helicoidally shaped way.

Example 12 is an example in concordance with Example 1 or with Example 1 and any one or more other Examples disclosed herein where the multi solenoid emitters magnetic field produced by the device, in combination with the application of electromagnetic radiative energy, produces selective molecular-type heating and molecular vibration and twisting to enhance a desired selective atomic reaction and in some cases, also a molecular chemical reaction.

Example 13 is an example in concordance with Example 1 or with Example 1 and any one or more other Examples disclosed herein where the multi solenoid emitters magnetic field produced by the device, in combination with the application of electromagnetic radiative energy, produces selective molecular type heating, molecular vibration, and twisting to enhance a desired molecular therapeutic biochemical reaction.

Example 14 is an example in concordance with Example 1 or with Example 1 and any one or more other Examples disclosed herein where the multi solenoid emitters magnetic field produced by the device, in combination with the application of electromagnetic radiative energy at the Larmor's frequency of the target atom, produces selective atomic heating and vibration to enhance a desired molecular therapeutic biochemical reaction.

Example 15 is an example in concordance with Example 1 or with Example 1 and any one or more other Examples disclosed herein where the multi solenoid emitters magnetic field produced by the device, in combination with the application of electromagnetic radiative energy at the Larmor's frequency of the target molecule, produces selective molecular heating, vibration, and twisting to enhance a desired molecular therapeutic biochemical reaction.

U.S. Provisional Patent Application No. 63/387,695, filed Dec. 15, 2022, is incorporated herein by reference, in its entirety.

In the description herein, specific details are set forth in order to provide a thorough understanding of the various example embodiments. It should be appreciated that various modifications to the embodiments will be readily apparent to those skilled in the art, and the generic principles defined herein may be applied to other embodiments and applications without departing from the spirit and scope of the disclosure. Moreover, in the following description, numerous details are set forth for the purpose of explanation. However, one of ordinary skill in the art should understand that embodiments may be practiced without the use of these specific details. In other instances, well-known structures and processes are not shown or described in order to avoid obscuring the description with unnecessary detail. Thus, the present disclosure is not intended to be limited to the embodiments shown but is instead to be accorded the widest scope consistent with the principles and features disclosed herein. Hence, these and other changes can be made to the embodiments in light of the above-detailed description. In general, in the following claims, the terms used should not be construed to limit the claims to the specific embodiments disclosed in the specification but should be construed to include all possible embodiments along with the full scope of equivalents to which such claims are entitled. Accordingly, the claims are not limited by the disclosure.

1. A neuromodulation device that employs Far-Field Magnetic Transfer effect comprising:
at least a pair of iterative multiple-magnet arrays where each array includes a number of magnets consecutively nested into bigger magnets with seemingly parallel walls in a multilayered fashion in order to better produce the Far-Field Magnetic Transfer effect.

2. The neuromodulation device of claim 1, further comprising at least a pair of iterative multiple-solenoid arrays wherein each array includes a number of solenoids that are independently electrically fed and consecutively nested into a bigger solenoid with seemingly parallel loops.

3. The neuromodulation device of claim 2, wherein an emitter array and receiver arrays are coupled to better produce the Far-Field Magnetic Power Transfer effect because each emitter solenoid is electrically fed with the same electrical signal frequency than its equivalent layer solenoid on the receiver multilayered array.

4. The neuromodulation device of claim 3, wherein both arrays contain a metallic core composed of iron, Permalloy, Mu-Metal or any metallic alloy with enough magnetic permeability to increase inductance.

5. The neuromodulation device of claim 4, wherein the magnetic power is applied directly to neuronal tissue in order to alter the electric signaling of neural circuits such as the Supra-Orbital cortex with the Accumbens Nucleus circuit.

6. The neuromodulation device of claim 5, wherein the application of the magnetic power is applied directly to neuronal tissue in order to stimulate the electric signaling of a neural circuit as the Prefrontal cortex with the Subthalamic Nucleus circuits.

7. The neuromodulation device of claim 6, wherein the emitter array and the receiver arrays are electrically connected in serial fashion and the solenoids on each array are connected in electrically parallel fashion in order to very easily and un-expensively control the device parameters.

8. The neuromodulation device of claim 6, wherein each emitter solenoid is electrically fed independently in order to send multiple signal variations to the solenoids, consequently producing a complex multi emitter magnetic field in order to generate a complex data input transducer capable of interacting with a much bigger neuronal population in a concentrated region of the brain,

9. The neuromodulation device of claim 8, wherein each receiver solenoid is electrically independently connected in order retrieve the signal variations collected by each solenoid of the receiver array, in order to generate a complex data output transducer capable of sensing a much bigger neuronal population in a concentrated region of the brain.

10. The neuromodulation device of claim 9, further comprising a catheter adapted to be inserted on the brain, the catheter comprising at least two mini multi-solenoids arrays in order to produce a Multi Emitter Magnetic Field in a particular region of the brain.

11. The neuromodulation device of claim 10, wherein the catheter deploys an helicoidally shaped coil that emerges from a deeper end and it is introduced through parenchyma by rotatory motion where the catheter contains at least two multi solenoid arrays in order to produce a Multi Emitter Magnetic Field in a wider and helicoidally shaped way.

12. The neuromodulation device of claim 11, wherein the multi solenoid emitters magnetic field produced by the device, in combination with the application of electromagnetic radiative energy, produces selective molecular type heating and molecular vibration and twisting in order to enhance a desired selective atomics as well as molecular chemical reaction.

13. The neuromodulation device of claim 11, wherein the multi solenoid emitters magnetic field produced by the device, in combination with the application of electromagnetic radiative energy, produces selective molecular type heating and molecular vibration and twisting in order to enhance a desired molecular therapeutic biochemical reaction.

14. The neuromodulation device of claim 11, wherein the multi solenoid emitters magnetic field produced by the device, in combination with the application of electromagnetic radiative energy, at the Larmor's frequency of the target atom that produces selective atomic heating and vibration in order to enhance a desired molecular therapeutic biochemical reaction.

15. The neuromodulation device of claim 14, wherein the multi solenoid emitters magnetic field produced by the device, in combination with the application of electromagnetic radiative energy, at the Larmor's frequency of the target molecule that produces selective molecular heating, vibration and twisting in order to enhance a desired molecular therapeutic biochemical reaction.
